(19) United States
(12) Patent Application Publication
(10) Pub. No.: US 2025/0249233 A1
(43) Pub. Date: Aug. 7, 2025

(54) PANTS FOR REPAIRING PELVIC FLOOR MUSCLES

(71) Applicant: Xiamen Solex High-Tech Industries Co., Ltd., Xiamen (CN)

(72) Inventors: Lixia WANG, Xiamen (CN); Chunjie HONG, Xiamen (CN)

(21) Appl. No.: 18/658,080

(22) Filed: May 8, 2024

(30) Foreign Application Priority Data
Feb. 6, 2024 (CN) 202410171634.7

(51) Int. Cl. A61N 1/04; A61N 1/36

(52) U.S. Cl. CPC A61N 1/0452; A61N 1/0476; A61N 1/048; A61N 1/0484; A61N 1/36034

(57) ABSTRACT

Pants for repairing pelvic floor muscles include a wearing pants body and an electrical stimulation member disposed on the wearing pants body; the electrical stimulation member is disposed on a crotch position of the wearing pants body corresponding to a perineum of a user; and the electrical stimulation member comprises a first flexible electrode and a second flexible electrode, and the first flexible electrode and the second flexible electrode respectively correspond to front and rear sides of the pelvic floor muscles of a user to apply electrical stimulation to the pelvic floor muscles.

### RELATED APPLICATIONS

This application claims priority to Chinese patent application number 202410171634.7, filed on Feb. 6, 2024. Chinese patent application number 202410171634.7 is incorporated herein by reference.

### FIELD OF THE DISCLOSURE

The present disclosure relates to pants for repairing pelvic floor muscles.

### BACKGROUND OF THE DISCLOSURE

Pelvic floor dysfunction diseases in females are a series of diseases of pelvic organ prolapse caused by dysfunction of the pelvic floor support structure due to various factors. As China enters an aging society, the pelvic floor dysfunction diseases are increasing year by year, seriously affecting life qualities of elderly female, and can easily cause mental and psychological problems. The diseases have gradually been taken into account, and pelvic floor muscle exercise is an emerging activity in the field of obstetrics and gynecology in recent years and acts as important driving forces in improving female pelvic floor function.

For females, the pelvic floor muscles support many organs in the pelvic cavity, such as the bladder, uterus, small intestine, rectum, and normal functions of these organs are maintained through a contraction of a group of the pelvic floor muscles. The pelvic floor muscles have powerful functions, but are easily injured, resulting in females suffering from the pelvic floor muscle dysfunction diseases. The function of the pelvic floor muscles will be affected by an increase in intra-abdominal pressure caused by pregnancy, childbirth, surgery, coughing, etc., resulting in incontinence, uterine prolapse, decreasing quality of sexual life, etc. Therefore, females need to perform rehabilitation trainings of the pelvic floor muscles to protect the function of the pelvic floor muscles after childbirth.

Some solutions using smart pants for repairing the pelvic floor muscles have been emerged on the market. Electrodes in the pants for repairs are disposed on inner thighs, repairing positions are inaccurately positioned so as to act on the inner thighs or other locations without pelvic floor muscles, and repair effects are poor. In some other pants for repairs, positive and negative conductive fabrics are arranged left and right, so that fabrics are easily wrinkled by personal behaviors due to the fabrics on the crotch area being too much and too wide, resulting in a product failure caused by contact short-circuits of the positive and negative electrodes.

### BRIEF SUMMARY OF THE DISCLOSURE

An objective of the present disclosure is to overcome the deficiencies in the existing techniques and provides pants for repairing pelvic floor muscles.

In order to solve the aforementioned technical problem, the present disclosure provides pants for repairing pelvic floor muscles, the pants for repairing pelvic floor muscles comprises a wearing pants body and an electrical stimulation member disposed on the wearing pants body, the electrical stimulation member is disposed on a crotch position of the wearing pants body, the electrical stimulation member comprises a first flexible electrode and a second flexible electrode, and the first flexible electrode and the second flexible electrode respectively correspond to front and rear sides of the pelvic floor muscles of a user to apply electrical stimulation to the pelvic floor muscles.

In a preferred embodiment, the crotch position of the wearing pants body is divided into a front section and a rear section, the first flexible electrode is disposed on the front section, and the second flexible electrode is disposed on the rear section.

In a preferred embodiment, the first flexible electrode covers the front section, and the second flexible electrode covers the rear section.

In a preferred embodiment, the first flexible electrode at least partially extends into the rear section, and the second flexible electrode at least partially extends into the front section.

In a preferred embodiment, the crotch position of the wearing pants body further comprises a stay-away section, the first flexible electrode and the second flexible electrode stay away from the stay-away section, and the stay-away section corresponds to clitoris and anus of the user.

In a preferred embodiment, the first flexible electrode and the second flexible electrode are detachably disposed on the wearing pants body.

In a preferred embodiment, the first flexible electrode and the second flexible electrode are respectively connected to the wearing pants body by magnetic attraction or glue.

In a preferred embodiment, the wearing pants body comprises a first annular connecting member and a second annular connecting member, the first annular connecting member is connected to the first flexible electrode, and the second annular connecting member is connected to the second flexible electrode.

In a preferred embodiment, the electrical stimulation member further comprises a controller disposed on the wearing pants body, the controller is configured to produce microcurrent stimulation on the pelvic floor muscles by the first flexible electrode and the second flexible electrode, the electrical stimulation member further comprises a first electrical connecting member and a second electrical connecting member, the first electrical connecting member and the second electrical connecting member are connected to the controller, the first flexible electrode is detachably connected to the first electrical connecting member, and the second flexible electrode is detachably connected to the second electrical connecting member.

In a preferred embodiment, the first flexible electrode and the first electrical connecting member are connected through a first metal four-piece buckle, and the second flexible electrode and the second electrical connecting member are connected through a second metal four-piece buckle.

In a preferred embodiment, the first electrical connecting member and the second electrical connecting member are connected to the controller through a conductive coating layer printed on the wearing pants body, or the first electrical connecting member and the second electrical connecting member are connected to the controller through wires or flexible conductive silicone.

In a preferred embodiment, the controller is detachably connected to the wearing pants body.

In a preferred embodiment, the first flexible electrode and the second flexible electrode are made of silver-fiber conductive cloth.

In a preferred embodiment, the first flexible electrode and the second flexible electrode are made of a conductive coating layer printed on the wearing pants body; or the first electrical connecting member and the second electrical connecting member are connected to a controller by flexible conductive silicone.

In a preferred embodiment, the first flexible electrode and the second flexible electrode form a plurality of transverse grooves separately disposed along a front and rear direction.

Compared with the existing techniques, the technical solution of the present disclosure has the following advantages.

The first flexible electrode and the second flexible electrode respectively correspond to the front and rear sides of the pelvic floor muscles of the user to apply the electrical stimulation to the pelvic floor muscles. Compared with wearable devices on the market that provide two electrodes corresponding to a right side and a left side of the pelvic floor muscles, the pants for repairing the pelvic floor muscles in the present disclosure provide the first flexible electrode and the second flexible electrode disposed on the front and rear sides of the crotch position, and a failure caused by contact between positive and negative electrodes will not occur. At the same time, this design can accurately position the first flexible electrode and the second flexible electrode at a position of the pelvic floor muscles for targeted repair, a treatment area is large, a repairing range is wide, and a repairing effect is good. The pelvic floor muscles are long-strip muscles extending in a front and rear direction, and compared with arranging the two electrodes (i.e., the first flexible electrode and the second flexible electrode) on left and right sides of the crotch position, in which a repairing range of the two electrodes on the left and right sides (i.e., a length of current pathway) is smaller, the first flexible electrode and the second flexible electrode are disposed on the front and rear sides of the crotch position in the present disclosure, and the repairing range is wider and the repairing effect is better.

The crotch position of the wearing pants body further comprises the stay-away section, and the first flexible electrode and the second flexible electrode stay away from the stay-away section. The stay-away section corresponds to clitoris and anus of the user. The arrangement of the stay-away section can prevent the clitoris and anus of the user from current stimulation.

### BRIEF DESCRIPTION OF THE DRAWINGS

FIG. 1 is a diagrammatic view of a crotch position of a wearing pants body of pants for repairing pelvic floor muscles in a preferred embodiment of the present disclosure.

FIG. 2A is a diagrammatic view of a maximum working distance of the pants for repairing the pelvic floor muscles in the preferred embodiment of the present disclosure.

FIG. 2B is a diagrammatic view of a maximum working distance of the pants for repairing pelvic floor muscles in the existing techniques.

FIG. 3 is a diagrammatic view of disassembly and assembly between the wearing pants body of the pants for repairing the pelvic floor muscles and a first flexible electrode and a second flexible electrode in the preferred embodiment of the present disclosure.

FIG. 4 is a diagrammatic view of an action between the first flexible electrode or the second flexible electrode and the pelvic floor muscles in the preferred embodiment of the present disclosure.

FIG. 5 is a diagrammatic view of the first flexible electrode partially extending into a rear section and the second flexible electrode partially extending into a front section to define a staggered structure in the present disclosure.

### DETAILED DESCRIPTION OF THE EMBODIMENTS

The present disclosure will be further described below in combination with the accompanying drawings and specific embodiments.

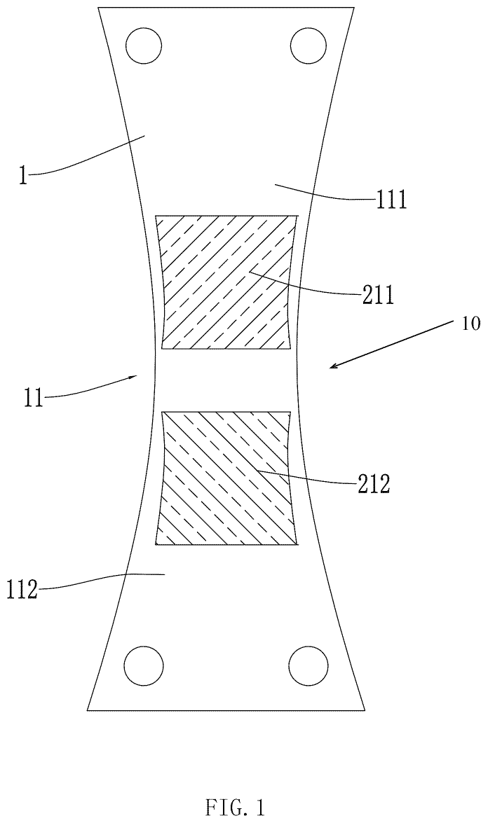

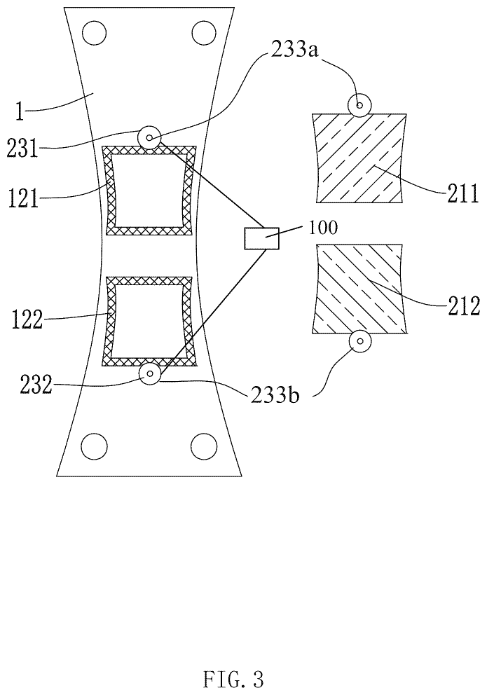

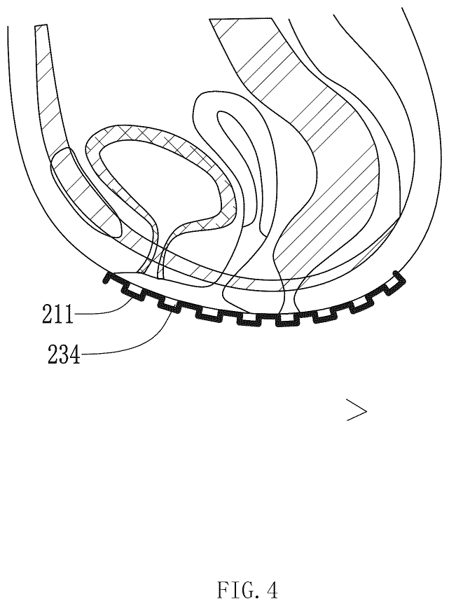

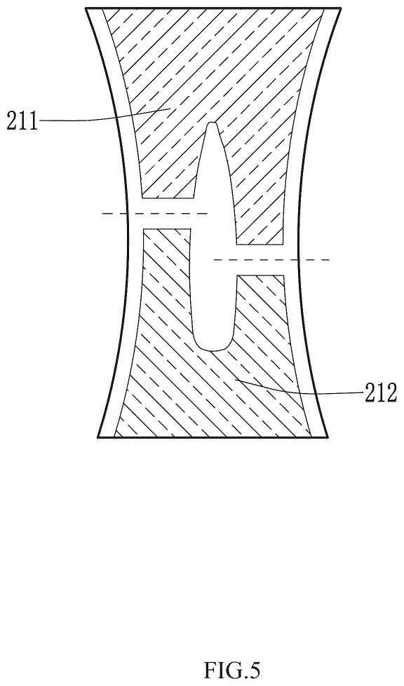

Referring to FIGS. 1-5, pants for repairing pelvic floor muscles comprise a wearing pants body 1 and an electrical stimulation member 10 disposed on the wearing pants body 1. The electrical stimulation member 10 is disposed on a crotch position 11 of the wearing pants body 1 corresponding to a perineum of a user. The crotch position 11 can be understood as a connection place of two pants legs, a top to a bottom of a middle part of the connection place can also be understood as a section of pants corresponding to thigh root and two-thigh intersection, i.e., a part where thighs are connected to a trunk. The electrical stimulation member 10 comprises a first flexible electrode 211 and a second flexible electrode 212, and the first flexible electrode 211 and the second flexible electrode 212 respectively correspond to a front side and a rear side of the pelvic floor muscles of the user to apply electrical stimulation to the pelvic floor muscles. The wearing pants body 1 may be in a shape of boxer briefs, etc., which is convenient to wear for the user when going out.

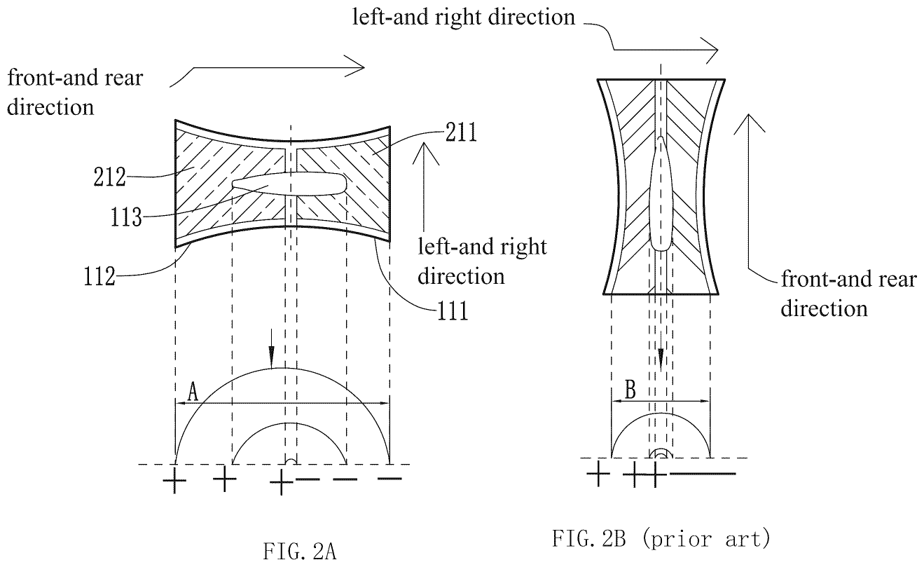

Compared with wearable devices on the market that provide two electrodes corresponding to a right side and a left side of the pelvic floor muscles, the pants for repairing the pelvic floor muscles in the present disclosure provides the first flexible electrode 211 and the second flexible electrode 212 disposed on the front and rear sides of the crotch position 11, and a failure caused by contact between positive and negative electrodes will not occur. At the same time, this design can accurately position the first flexible electrode and the second flexible electrode at a position of the pelvic floor muscles for targeted repairs. A treatment area is large, a repairing range is wide, and repairing effect is good. The pelvic floor muscles are long-strip muscles extending in a front and rear direction, and compared with arranging the two electrodes (i.e., the first flexible electrode 211 and the second flexible electrode 212) on left and right sides of the crotch position 11, in which a repairing range of the two electrodes on the left and right sides (i.e., a length of current pathway) is smaller, the first flexible electrode 211 and the second flexible electrode 212 are disposed on the front and rear sides of the crotch position 11 in the present disclosure, and the repairing range is wider and the repairing effect is better. Take a maximum working distance of positive and negative electrodes under microcurrent as an example. Referring to FIG. 2A, in the present disclosure, a maximum working distance of a front and rear electrode placement is A. In the existing techniques, referring to FIG. 2B, a maximum working distance of a left and right electrode placement is B. As shown in FIGS. 2A and 2B, three points, maximum, minimum, and middle distances under the microcurrent are respectively selected. As a result, A is much larger than B. It can be seen that the repairing range of the front and rear electrode placement is wider, an effective area applied to the pelvic floor muscles is also larger, and the repairing effect is better.

In this embodiment, the crotch position 11 of the wearing pants body 1 is divided into a front section 111 and a rear section 112. The first flexible electrode 211 is disposed on the front section 111, and the second flexible electrode 212 is disposed on the rear section 112. The front section 111 and the rear section 112 are separated by a preset distance in the front and rear direction using a transverse midline of the crotch position 11 as a boundary.

In this embodiment, the first flexible electrode 211 covers the front section 111, and the second flexible electrode 212 covers the rear section 112. In some simple replacements, the first flexible electrode 211 at least partially extends into the rear section 112, and the second flexible electrode 212 at least partially extends into the front section 111 to define a staggered structure.

In some simple replacements, the crotch position 11 of the wearing pants body 1 further comprises a stay-away section 113, and the first flexible electrode 211 and the second flexible electrode 212 stay away from the stay-away section 113.

The stay-away section 113 corresponds to clitoris and anus of the user. An arrangement of the stay-away section 113 can prevent the clitoris and anus of the user from current stimulation.

In this embodiment, the first flexible electrode 211 and the second flexible electrode 212 are detachably disposed on the wearing pants body 1. The first flexible electrode 211 and the second flexible electrode 212 are respectively connected to the wearing pants body 1 by magnetic attraction or glue. Specifically, the wearing pants body 1 comprises a first annular connecting member 121 and a second annular connecting member 122. The first annular connecting member 121 is connected to the first flexible electrode 211, and the second annular connecting member 122 is connected to the second flexible electrode 212. The first annular connecting member 121 and the second annular connecting member 122 may be an annular rubber magnet sewn by a sewing process for the magnetic attraction. Edges of the first flexible electrode 211 and the second flexible electrode 212 have magnets or ferromagnetic materials. Alternatively, the first annular connecting member 121 and the second annular connecting member 122 are annular double-sided tape for fixing the first flexible electrode 211 and the second flexible electrode 212 by bonding.

The electrical stimulation member 10 further comprises a controller 100 disposed on the wearing pants body 1, and the controller 100 is used to produce microcurrent stimulation on the pelvic floor muscles by the first flexible electrode 211 and the second flexible electrode 212. The electrical stimulation member 10 further comprises a first electrical connecting member 231 and a second electrical connecting member 232. The first electrical connecting member 231 and the second electrical connecting member 232 are connected to the controller 100. The first flexible electrode 211 is detachably connected to the first electrical connecting member 231, and the second flexible electrode 212 is detachably connected to the second electrical connecting member 232. Specifically, the first flexible electrode 211 and the first electrical connecting member 231 are connected through a first metal four-piece buckle 233a, and the second flexible electrode 212 and the second electrical connecting member 232 are connected through a second metal four-piece buckle 233b. The first flexible electrode 211 and the second flexible electrode 212 can be easily disassembled and assembled on the crotch position 11.

In this embodiment, the first electrical connecting member 231 and the second electrical connecting member 232 are connected to the controller 100 through a conductive coating layer printed on the wearing pants body 1, and the conductive coating layer can prevent discomfort caused by wires protruding from a surface of underwear (i.e., the wearing pants body 1). In some alternatives, the first electrical connecting member 231 and the second electrical connecting member 232 are connected to the controller 100 through wires or flexible conductive silicone.

In this embodiment, the controller 100 is detachably connected to the wearing pants body 1. For example, the controller 100 can be attached to an outside of the wearing pants body 1 by hook and loop, etc.

In this embodiment, the first flexible electrode 211 and the second flexible electrode 212 are made of silver-fiber conductive cloth, or the first flexible electrode 211 and the second flexible electrode 212 are made of the flexible conductive silicone. The first flexible electrode 211 and the second flexible electrode 212 form a plurality of transverse grooves 234 separately disposed along the front and rear direction. A cloth of the wearing pants body 1 is ultra-fine nylon or other skin-friendly fabrics.

In some alternative embodiments, the first flexible electrode 211 and the second flexible electrode 212 are made of a conductive coating layer printed on the wearing pants body; or the first electrical connecting member 231 and the second electrical connecting member 232 are connected to the controller 100 by flexible conductive silicone.

The advantages of the plurality of transverse grooves 234 are as follows. 1. Accurate fit: the first flexible electrode 211 and the second flexible electrode 212 are formed by long strips using an embossing process, is elastic and close-fitting, has a staggered structure having a rough feeling, enables the fabric to be easily bent and deformed following a shape of the pelvic floor muscles, has a given sense of support, can replace a role of supporting cotton, etc., can achieve precise fit on skin, cause no stinging sensation stimulated by stronger local microcurrent, is breathable and comfortable, can provide the most effective and comfortable repairing experience for the user, and provides the user with a very good experience. 2. Effective water locking: \textcircled{1} Material: The silver-fiber conductive cloth usually consists of silver fibers and base materials, and the silver fibers have excellent conductivity and water absorption properties. \textcircled{2} Shape: a main function of the long-strip rough design is to increase a contact area and a frictional force. When water is sprayed on the silver-fiber conductive cloth, water molecules contact the plurality of transverse grooves 234, due to a special shape and a rough surface thereof, the water molecules are adsorbed in the plurality of transverse grooves 234, more water will be stored and be not easily volatilized due to a temperature of the water being lower compared with a flat surface, so that water is effectively locked. \textcircled{3} Viscosity force: A surface of the long-strip rough structure has viscosity properties, i.e., the surface has an adhesion effect on liquid, and the viscosity force can resist flow of the liquid, resulting in forming certain retention of the liquid on the surface of the structure. \textcircled{4} Capillarity effect: the capillarity effect refers to the flow of the liquid in small pipes caused by surface tension. In the long-strip rough structure, the liquid forms a series of small passages on the surface of the structure due to tiny grooves and protrusions on the surface of the structure, so that the capillarity effect is produced. \textcircled{5} Self-attractive force: the self-attractive force refers to an attractive force of an object generated due to gravity. In the long-strip rough structure, the liquid forms a certain gravity distribution on the surface of the structure due to the tiny grooves and protrusions on the surface of the structure, thus producing the self-attractive force.

The aforementioned embodiments are merely some embodiments of the present disclosure, and the concepts of the disclosure are not limited thereto. Thus, it is intended that the present disclosure cover non-substantive modifications of the present disclosure provided they are made based on the concepts within the technical scope disclosed in the present disclosure by any technical person skilled in the art.

What is claimed is:

1. Pants for repairing pelvic floor muscles, comprising:
a wearing pants body, and
an electrical stimulation member disposed on the wearing pants body, wherein:
the electrical stimulation member is disposed on a crotch position of the wearing pants body,
the electrical stimulation member comprises a first flexible electrode and a second flexible electrode, and
the first flexible electrode and the second flexible electrode respectively correspond to front and rear sides of the pelvic floor muscles of a user to apply electrical stimulation to the pelvic floor muscles.

2. The pants for repairing the pelvic floor muscles according to claim 1, wherein:
the crotch position of the wearing pants body is divided into a front section and a rear section,
the first flexible electrode is disposed on the front section, and
the second flexible electrode is disposed on the rear section.

3. The pants for repairing the pelvic floor muscles according to claim 2, wherein:
the first flexible electrode covers the front section, and
the second flexible electrode covers the rear section.

4. The pants for repairing the pelvic floor muscles according to claim 3, wherein:
the first flexible electrode at least partially extends into the rear section, and
the second flexible electrode at least partially extends into the front section.

5. The pants for repairing the pelvic floor muscles according to claim 1, wherein:
the crotch position of the wearing pants body further comprises a stay-away section,
the first flexible electrode and the second flexible electrode stay away from the stay-away section, and
the stay-away section corresponds to clitoris and anus of the user.

6. The pants for repairing the pelvic floor muscles according to claim 1, wherein:
the first flexible electrode and the second flexible electrode are detachably disposed on the wearing pants body.

7. The pants for repairing the pelvic floor muscles according to claim 6, wherein:
the first flexible electrode and the second flexible electrode are respectively connected to the wearing pants body by magnetic attraction or glue.

8. The pants for repairing the pelvic floor muscles according to claim 6, wherein:
the wearing pants body comprises a first annular connecting member and a second annular connecting member,
the first annular connecting member is connected to the first flexible electrode, and
the second annular connecting member is connected to the second flexible electrode.

9. The pants for repairing the pelvic floor muscles according to claim 6, wherein:
the electrical stimulation member further comprises a controller disposed on the wearing pants body,
the controller is configured to produce microcurrent stimulation on the pelvic floor muscles by the first flexible electrode and the second flexible electrode,
the electrical stimulation member further comprises a first electrical connecting member and a second electrical connecting member,
the first electrical connecting member and the second electrical connecting member are connected to the controller,
the first flexible electrode is detachably connected to the first electrical connecting member, and
the second flexible electrode is detachably connected to the second electrical connecting member.

10. The pants for repairing the pelvic floor muscles according to claim 9, wherein:
the first flexible electrode and the first electrical connecting member are connected through a first metal four-piece buckle, and
the second flexible electrode and the second electrical connecting member are connected through a second metal four-piece buckle.

11. The pants for repairing the pelvic floor muscles according to claim 9, wherein:
the first electrical connecting member and the second electrical connecting member are connected to the controller through a conductive coating layer printed on the wearing pants body, or
the first electrical connecting member and the second electrical connecting member are connected to the controller through wires or flexible conductive silicone.

12. The pants for repairing the pelvic floor muscles according to claim 9, wherein:
the controller is detachably connected to the wearing pants body.

13. The pants for repairing the pelvic floor muscles according to claim 1, wherein:
the first flexible electrode and the second flexible electrode are made of silver-fiber conductive cloth.

14. The pants for repairing the pelvic floor muscles according to claim 9, wherein:
the first flexible electrode and the second flexible electrode are made of a conductive coating layer printed on the wearing pants body; or
the first electrical connecting member and the second electrical connecting member are connected to a controller by flexible conductive silicone.

15. The pants for repairing the pelvic floor muscles according to claim 1, wherein:
the first flexible electrode and the second flexible electrode form a plurality of transverse grooves separately disposed along a front and rear direction.
